(19) United States
(12) Patent Application Publication
(10) Pub. No.: US 2023/0386104 A1
(43) Pub. Date: Nov. 30, 2023

(54) INFORMATION DISPLAY DEVICE AND INFORMATION DISPLAY METHOD

(71) Applicant: MAXELL, LTD., Kyoto (JP)

(72) Inventors: Koji KANIWA, Kyoto (JP); Tamotsu ITO, Kyoto (JP)

(21) Appl. No.: 18/033,091

(22) PCT Filed: Oct. 23, 2020
(86) PCT No.: PCT/JP2020/039934

§ 371 Date: Apr. 21, 2023

(51) Int. Cl. G06T 11/20; G06F 3/14; G10L 15/26; A61B 5/16; A61B 5/00

(52) U.S. Cl. CPC G06T 11/206; G06F 3/14; G10L 15/26; A61B 5/165; A61B 5/6898; A61B 2503/12

(57) ABSTRACT

An information display device comprises a communication device connected to a communication network; a display; and a processor connected to each of the communication device and the display. The processor is configured to: calculate an information fake level indicating a level of credibility of display information received by the communication device via the communication network and displayed on the display based on a characteristic relating to the credibility on the display information; calculate a user-specific fake level coefficient indicating a level in which a user who operates the information display device is able to judge accuracy of the credibility of the display information based on an index representing how the user tends to judge the credibility; correct the information fake level using the user-specific fake level coefficient to calculate a user-specific fake level; and display the user-specific fake level on the display.

### TECHNICAL FIELD

The present invention relates to an information display device and an information display method, and more particularly relates to a technique for providing a user with the credibility of information.

### BACKGROUND ART

In recent years, mobile information terminals (information display devices), typically smartphones, have been widely used. A user of a smartphone can get various types of information from external servers through a telephone line or a LAN (Local Area Network) by connecting his or her smartphone to the internet. Furthermore, in a smartphone that is a personal information terminal, an individual user who operates the smartphone can obtain information about himself or herself by means of application software, various sensors, and the like.

As a system for identifying the credibility of news that is available on the internet, Patent Literature 1 discloses a system in which, “the received news content may be evaluated to identify an associated media outlet, an associated journalist, and at least one associated predefined topic. A current score for each of the associated media outlet, the associated journalist, and the at least one associated predefined topic may be identified based on the stored information. A veracity score may be scored based on the identified current scores for the associated media outlet, the associated journalist, and the at least one associated predefined topic. A display associated with the received news content may be modified based on the generated veracity score (excerpted from Abstract)”.

### CITATION LIST

### Patent Literature

Patent Literature 1: JP-A-2020-508518

### SUMMARY OF INVENTION

### Technical Problem

The information available on the internet includes not only content such as news, but also content issued by persons through SNS. In case that whether these kinds of information available through the internet are credible or not is uncertain, there is a risk that such information deceives a reader who has been in contact with the information. In addition, there may be a further risk that a bona fide person who has received information with less credibility will spread it on SNS.

On the other hand, as in the same as that among readers who have been in contact with the same information, some readers are deceived by the information while other readers are not, whether a reader is deceived or not by certain content depends not only on the credibility of the content per se, but also on how much attention the reader pays to the credibility of the content.

The system according to Patent Literature 1 can identify the credibility of news content per se, however, does not consider a level of risk that an individual reader who has been in contact with the news content believes it. Furthermore, the system according to Patent Literature 1 can identify the credibility of news content whose source medium or journalist who wrote the news are known, however, cannot identify the credibility of information issued by ordinary persons or anonymous information, such as the content shared on SNS. Thus, such problems still remain therein.

In view of the above, an object of the present invention is to provide information relating to not only the credibility of news content, but also the credibility of information acquired through public networks such as the internet and broadcast networks (this information thus received is to be displayed on a display screen of an information display device, and accordingly, hereinafter, it is referred to as “display information”), while taking into consideration a state of a reader.

### Solution to Problem

In order to solve the problems above, the present invention includes the features described in the scope of claims. One of the aspects thereof is an information display device comprising: a communication device connected to a communication network; a display; and a processor connected to each of the communication device and the display, the processor being configured to: calculate an information fake level indicating a level of credibility of display information that is received by the communication device via the communication network and displayed on the display based on a characteristic relating to the credibility on the display information; calculate a user-specific fake level coefficient indicating a level in which a user who operates the information display device is able to judge accuracy of the credibility of the display information based on an index representing how the user tends to judge the credibility; correct the information fake level using the user-specific fake level coefficient to calculate a user-specific fake level; and display the user-specific fake level on the display.

### Advantageous Effects of Invention

According to the present invention, it is possible to provide information relating to not only the credibility of news content, but also the credibility of information acquired through public networks such as the internet and broadcast networks, while taking into consideration a state of a reader. The problems, configurations, and advantageous effects other than those described above will be clarified by explanation of the embodiments below.

### BRIEF DESCRIPTION OF DRAWINGS

FIG. 1 is a schematic diagram for explaining an outline of an information display device according to the present embodiment.

FIG. 2 is a hardware configuration diagram illustrating an example of an internal configuration of a smartphone.

FIG. 3 is a functional block diagram illustrating an example of a functional block configuration according to a first embodiment.

FIG. 4 illustrates a flowchart of a processing procedure of a fake level display process according to the first embodiment.

FIG. 5 illustrates a display example of display information according to the first embodiment.

FIG. 6 illustrates a display example by a user-specific fake level being calculated display process according to the first embodiment.

FIG. 7 illustrates a flowchart of a processing procedure of an information fake level calculating process according to the first embodiment.

FIG. 8 illustrates a flowchart of a processing procedure of personality information acquiring process according to the first embodiment.

FIG. 9 illustrates a user-specific fake level coefficient table according to the first embodiment.

FIG. 10 illustrates a display example of a user-specific fake level displaying process according to the first embodiment.

FIG. 11 is a schematic diagram for explaining an outline of a second embodiment.

FIG. 12 is a functional block diagram illustrating an example of a functional block configuration of the second embodiment.

FIG. 13 illustrates a flowchart of a processing procedure of a fake level display process according to the second embodiment.

FIG. 14 illustrates a flowchart of a processing procedure of a physical condition information acquiring process according to the second embodiment.

FIG. 15 illustrates a user-specific fake level coefficient table according to the second embodiment.

FIG. 16 is a functional block diagram illustrating an example of a functional block configuration according to a third embodiment.

FIG. 17 illustrates a flowchart of a processing procedure of a fake level display process according to the third embodiment.

FIG. 18 illustrates a user-specific fake level coefficient table according to the third embodiment.

FIG. 19 is a schematic diagram for explaining an outline of a fourth embodiment.

FIG. 20 is a functional block diagram illustrating an example of a functional block configuration according to a fourth embodiment.

FIG. 21 illustrates a flowchart of a processing procedure of a fake level display process according to the fourth embodiment.

### DESCRIPTION OF EMBODIMENTS

Hereinafter, embodiments of the present invention will be described with reference to the drawings. Throughout all the drawings, the common drawings are provided with the common reference signs, and repetitive explanation therefor will be omitted.

### First Embodiment

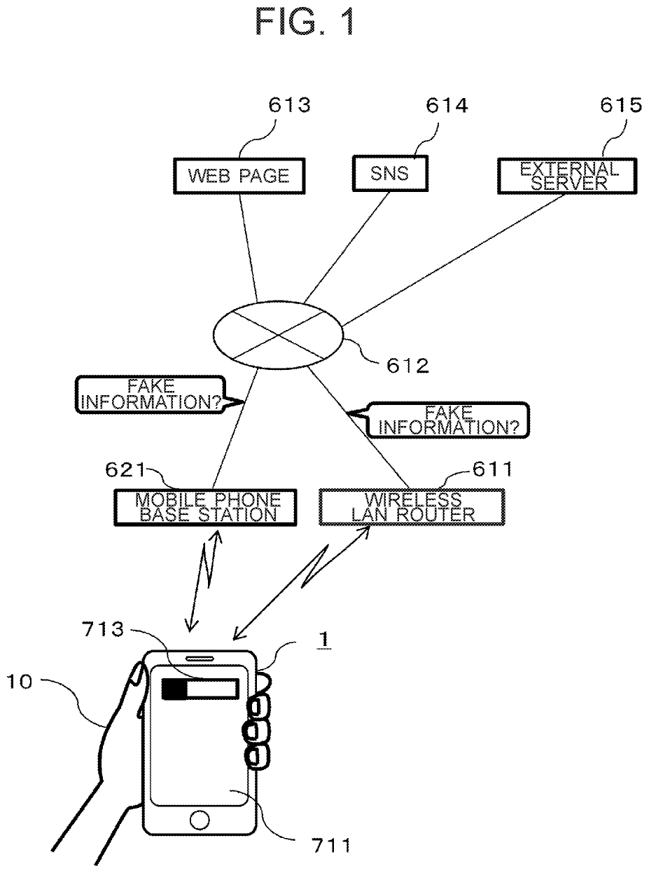

FIG. 1 is a schematic diagram for explaining an outline of an information display device according to the present embodiment.

FIG. 1 illustrates that a user 10 who is operating a smartphone 1, which is an information display device, is acquiring, through an internet 612, display information from a WEB (World Wide Web) page 613, the display information from SNS (Social Netwarking Service) 614, and the like to make it displayed on a display screen 711 of the smartphone 1. In the present embodiment, the smartphone 1 is exemplified as an information display device, however, the information display device may be a mobile information terminal such as a tablet or an information processing device such as a PC as long as it has a function of receiving and displaying display information from the internet 612 or the SNS 614.

The smartphone 1 may be connected to the internet 612, for example, via a mobile phone base station 621, which is a mobile phone network, or via a wireless LAN router (Wi-Fi (registered mark) router) 611. In both the approaches, in the sense of connecting to the internet 612, they mean the same as the background of the present embodiment.

The internet 612 is connected to an external server 615 that issues fake-related information. The smartphone 1 is configured to acquire, from the external server 615, fake-related information necessary for implementing the present embodiment. The external server 615 may be connected to a site for managing transmission of fake information, or may be an independent server for issuing fake-related information.

There is a possibility that display information with doubtful credibility is mixed among pieces of display information (display information from the WEB page 613, display information from the SNS 614, and the like) acquired through the internet 612 by the user 10 who operates the smartphone 1. (FIG. 1 illustrates it as “fake information?”.)

In the present embodiment, the accuracy of credibility is referred to as a “fake level”. A fake level means the opposite of accuracy of credibility (0% to 100%), in which the accuracy of credibility 100% corresponds to the fake level 0%, and accuracy of credibility 0% corresponds to the fake level 100%. That is, the fake level (%) is a value obtained by subtracting the accuracy (%) of credibility from 100%.

In the present embodiment, the smartphone 1 acquires fake-related information from the external server 615 that issues fake-related information, carries out internal processing of the smartphone 1 to calculate a fake level thereof. In the case of display information already provided with fake-related information on the display information itself, the smartphone 1 uses the display information as it is, naturally.

In the situation that the user 10 has been in contact with the display information acquired from the internet 612 or the SNS 614, even if the display information is the same as the one with which other users have been in contact, whether the user 10 may be deceived or not thereby depends on the personality of the user 10 and the knowledge that the user 10 has. That is, among the users, how they perceive a fake level of the display information differs, and thus, even when they are in contact with the same display information, some users are deceived thereby while other users are not.

In this respect, in the present embodiment, a user-specific fake level coefficient that indicates a level in which the user 10 who operates the smartphone 1 is able to judge the credibility of display information is calculated based on an index representing how the user 10 tends to judge the credibility, a fake level (information fake level) specific to the display information is corrected using the calculated coefficient, and the fake level is optimized taking into consideration personality information (gullibility) about the user. Herein, the fake level thus optimized corresponds to a user-specific fake level, which is the fake level to be displayed.

The information fake level is calculated using the characteristics relating to the credibility on the display information. Specifically, as the “characteristics relating to the credibility on the display information”, for example, at least one or any combination of the type of a site where the display information was made available, a person who wrote the display information, information indicating a person who made the display information available, evaluation information in which a person who made the display information available has been evaluated in terms of the credibility on the display information and the evaluation is provided, when the display information was spread through public networks, how fast the display information was spread through public networks, and sentence expressions included in the display information may be used. An information fake level may be calculated by the smartphone 1 based on the characteristics relating to the credibility on the display information, or an information fake level which has been already calculated may be acquired (reception is one aspect of acquisition) by the smartphone 1. In the case of acquiring the information fake level, the smartphone 1 can omit an information fake level calculating process from the processing which will be described later, and acquire the information fake level of the display information at the same time or at a different timing of receiving the display information.

The “index representing how a user tends to judge the credibility” used in calculation of a user-specific fake level coefficient is a value that varies depending on the gullibility of a user. It is considered that a non-careful user is more gullible than a user who is not. Furthermore, even the same user may be more likely to be deceived, when, for example, he or she is excited or in a hurry, or when he or she is in an emergency situation and thus is so upset. Considering the above, evaluation of a user in terms of gullibility may be prepared in advance and a result of the evaluation may be used as an index. Furthermore, if a user is excited or in a hurry, change in his or her physical conditions, such as increase in blood pressure, increase in heart rate, increase in respiration rate, and increase in body temperature are more likely to appear, and thus the physical condition information may be used as an index. Note that an embodiment in which the physical condition information is used as an index will be described in more detail in <Second Embodiment> to be described later.

In FIG. 1, a bar graph is used to display the fake level as determined in a fake level area 713 of the display screen 711. Modes for displaying a fake level will be described later.

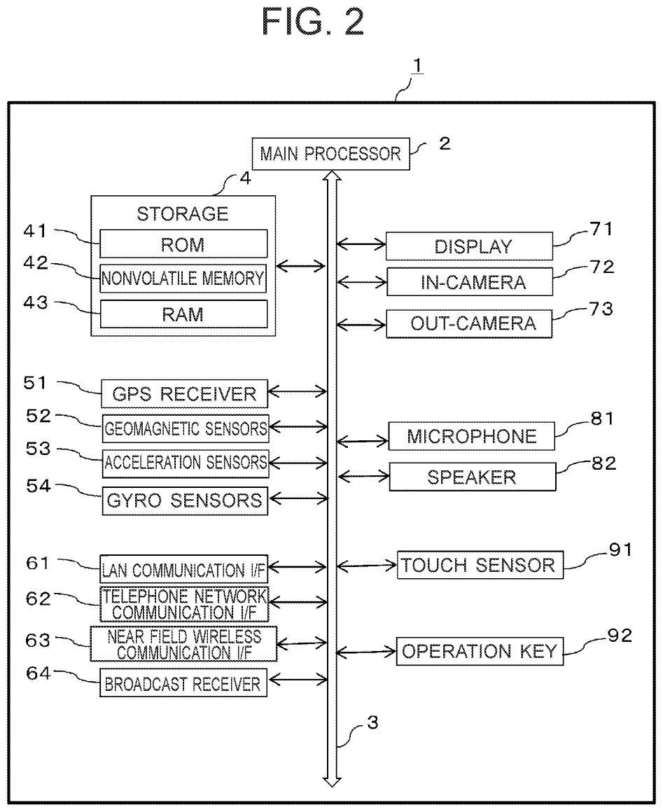

FIG. 2 is a hardware configuration diagram illustrating an example of an internal configuration of the smartphone 1.

The smartphone 1 includes a main processor 2, a storage 4, a GPS receiver 51, geomagnetic sensors 52, acceleration sensors 53, gyro sensors 54, a LAN communication I/F 61, a telephone network communication I/F 62, a near field wireless communication I/F 63, a broadcast receiver 64, a display 71, an in-camera 72, an out-camera 73, a microphone 81, a speaker 82, a touch sensor 91, and an operation key 92, and these components are connected so as to communicate with each other via a system bus 3.

The main processor 2 is a microprocessor unit that controls the overall operations of the smartphone 1 in accordance with predetermined operation programs.

The system bus 3 is a data communication channel for transmitting and receiving various types of commands, data, and the like between the main processor 2 and each configuration blocks in the smartphone 1.

The storage 4 includes a ROM 41 that stores therein programs or the like for controlling the operations of the smartphone 1, a nonvolatile memory 42 that stores therein various kinds of data such as operation setting values, detected values from each sensor, objects including content, library information downloaded from a library, and the like, and a rewritable RAM 43 such as a work area used in various program operations.

Furthermore, the storage 4 can store therein operation programs downloaded on networks, various kinds of data created by operation programs, and the like. The storage 4 can further store therein content such as moving images, still images, and sounds downloaded on networks. Still further, the storage 4 can store data of moving images and still images captured using an image capturing function of the in-camera 72 and out-camera 73. The storage 4 needs to hold stored information even while the smartphone 1 is not being supplied with power from the outside. Accordingly, for example, a device such as a semiconductor device memory such as a flash ROM or an SSD (Solid State Drive), a magnetic disk drive such as an HDD (Hard Disc Drive), or the like is used as the storage 4. Note that each of the operation programs stored in the storage 4 can be updated and expanded by downloading from the external server 615.

The smartphone 1 includes the GPS (Global Positioning System) receiver 51, the geomagnetic sensors 52, the accelerometer sensors 53, and the gyro sensors 54. Using these sensors enables detection of the position, tilting, orientation, motion, and the like of the smartphone 1. The smartphone 1 may further include other sensors such as an illuminance sensor, an altitude sensor, and a proximity sensor.

The LAN communication I/F 61 is connected to the internet 612 via access points and the like, and transmits and receives data to and from the external server 615 on the internet 612. Connection to the access points and the like may be realized by wireless communication such as Wi-Fi (registered trademark). (In the present embodiment, use of the wireless LAN router 611 is assumed.)

The telephone network communication I/F 62 carries out telephone communication (call) and transmission and reception of data by wireless communication with the mobile phone base station 621 and the like of a mobile phone communication network. Communication with the mobile phone base station 621 and the like may be realized by a W-CDMA (Wideband Code Division Multiple Access (registered trademark) system, a GSM (Global System for Mobile communications) system, an LTE (Long Term Evolution) system, or other communication systems.

The near field wireless communication I/F 63 exchanges data with an external Bluetooth (registered trademark) device and an external NFC device by Bluetooth (registered trademark) communication, NFC standard communication, or the like.

The broadcast receiver 64 receives broadcast signals of, for example, TV broadcasting and radio broadcasting.

Each of the LAN communication I/F 61, the telephone network communication I/F 62, the near field wireless communication I/F 63, and the broadcasting receiver 64 includes an encoding circuit, a decoding circuit, an antenna, and the like. The smartphone 1 may further include, in addition to the communication devices described above, other communication I/Fs such as an infrared communication I/F.

The display 71 is a display device such as a liquid crystal display with backlights or a self-luminous organic EL display, and provides the user 10 with display information such as image data and video data obtained on the internet 612, news content, and text information, or image information and video information captured by the in-camera 72 and out-camera 73 by displaying them thereon.

In the smartphone 1, the in-camera 72 is provided on the same surface as the surface on which the display 71 is provided. The in-camera 72 is used for capturing a face image of the user 10 for the purpose of identification of the user 10.

The out-camera 73 is provided on the rear surface of the smartphone 1. The out-camera 73 is used for imaging a scenery and the like.

Each of the in-camera 72 and the out-camera 73 is a camera that converts light received from a lens into an electric signal using an electronic device such as a CCD (Charge Coupled Device) or CMOS (Complementary Metal Oxide Semiconductor) sensor, thereby inputting image information about the surroundings and objects.

The microphone 81 converts the sounds in a real space, voices of a user, and the like into audio information and inputs the audio information.

The speaker 82 outputs audio information and the like necessary for the user. Needless to say, an earphone and a headphone can also be connected to the smartphone 1, and they can be selectively used depending on the purposes of use.

The touch sensor 91 is laminated on the display screen 711 of the display 71.

The operation key 92 is configured with arrangement of button switches and the like.

The touch sensor 91 and the operation key 92 are examples of an operation input device for inputting an operation instruction to the smartphone 1, and may be other operation input devices. Alternatively, a separate mobile terminal device connected to the smartphone 1 by wired or wireless communication may be used to operate the smartphone 1 via the LAN communication I/F 61 or the near field wireless communication I/F 63.

Furthermore, analyzing a video captured by the in-camera 72 and using an action such as a gesture may allow the user to operate the smartphone 1.

Note that the hardware configuration example of the smartphone 1 illustrated in FIG. 2 includes many components that are not essential to the present embodiment, and the advantageous effects of the present embodiment are not impaired even in a configuration in which these components are not provided. Furthermore, configurations which are not illustrated therein, for example, an electronic money payment function, may be further added in the configuration.

### (Functional Block According to Present Embodiment)

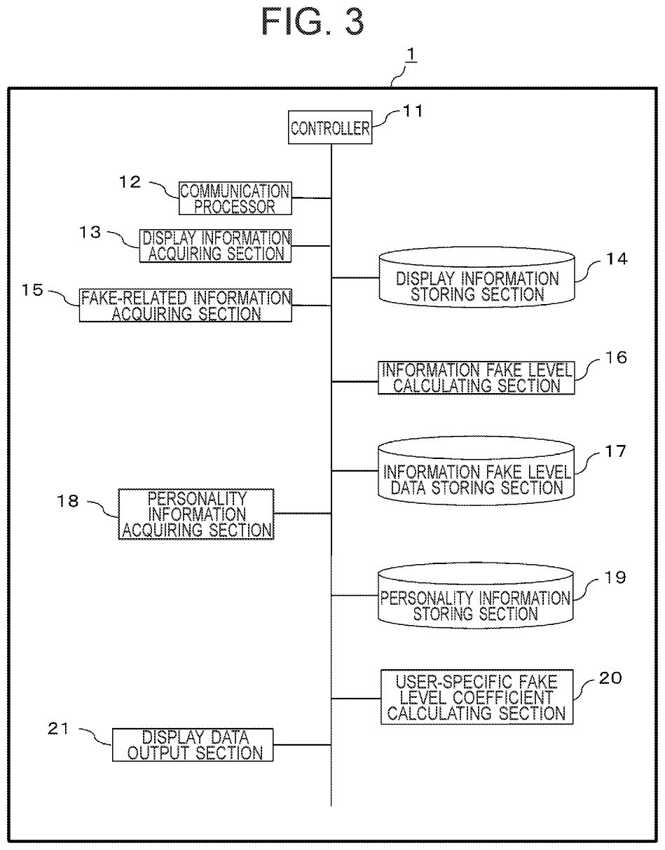

FIG. 3 is a functional block diagram illustrating an example of a functional block configuration according to the first embodiment.

A controller 11 is a function of controlling the whole operations of the smartphone 1, and mainly, the main processor 2 reads a program stored in the ROM 41 of the storage 4 and loads the program into the RAM 43, whereby the controller 11 is configured.

A communication processor 12 is a function of carrying out communication processing for connecting the smartphone 1 to the internet 612 by means of the LAN communication I/F 61 and the telephone network communication I/F 62.

A display information acquiring section 13 is a function of acquiring display information from the WEB page 613, the SNS 614, and the like through the internet 612 by means of the communication processor 12.

A display information storing section 14 is a function of storing display information acquired by the display information acquiring section 13 in the nonvolatile memory 42 of the storage 4.

A fake-related information acquiring section 15 is a function of acquiring information relating to fake from the external server 615 that issues fake-related information by means of the communication processor 12. The external server 615 verifies the display information from the WEB page 613 and SNS (fact checking), and issues fake-related information including a verification result. As the external server 615, for example, media partners of the Fact Check Initiative Japan (FIJ) may be used.

An information fake level calculating section 16 is a function of applying AI (Artificial Intelligence) inference to the fake-related information acquired by the fake-related information acquiring section 15 to calculate a fake level of the display information as inferred. In AI processing, the regularity of data and rules are learned to obtain inferences derived therefrom. The information fake level calculating section 16 calculates a fake level by the AI processing based on information relating to fake, past display information and the accuracy of credibility of the past display information, and the like.

When the fake-related information acquiring section could not acquire the fake information, the information fake level calculating section 16 uses past fake information in inference of a fake level by the AI processing. The information fake level calculating section 16 may be configured to calculate a fake level by conventional rule-based calculation processing without using the AI processing.

An information fake level data storing section 17 is a function of storing, in the nonvolatile memory 42 or the RAM 43 of the storage 4, the fake level (fake level relating to the display information, and is thus, hereinafter, referred to as “information fake level”) of the display information obtained by the information fake level calculating section 16, together with the corresponding display information.

A personality information acquiring section 18 is a function of acquiring information regarding the personality (gullibility) of the user 10 who operates the smartphone 1. The personality information acquiring section 18 asks various questions to the user 10 who operates the smartphone 1, and evaluates the personality including the gullibility of the user 10 based on the answers to the questions. The personality information acquiring section 18 converts a result of the personality evaluation into, for example, a score indicating the gullibility to generate personality information.

A personality information storing section 19 is a function of storing the information regarding the personality (gullibility) of the user 10 acquired by the personality information acquiring section 18 in the nonvolatile memory 42 of the storage 4 in association with information uniquely identifying the user 10 (user identification information). The user identification information may be physical feature information such as a face image of the user 10 captured by the in-camera 72, iris information, fingerprint information read using a fingerprint sensor (not illustrated), and voiceprint information of the user 10 collected using the microphone 81, or may be simply the name of the user 10.

The user-specific fake level coefficient calculating section 20 is a process of determining the personality information stored by the personality information storing section 19 and calculating a coefficient (weighting) for the information fake level obtained by the information fake level calculating section 16. A fake level to be displayed is determined by the user-specific fake level coefficient calculating section 20. The coefficient (weighting) for the information fake level will be described later.

A display data output section 21 is a function of displaying, on the display screen 711 of the smartphone 1, the display information stored in the nonvolatile memory 42 of the storage 4 by the display information storing section 14, the fake level (information specific to the user, and thus is, hereinafter, referred to as a “user-specific fake level”) generated in consideration of user personality information, and the like. The user-specific fake level to be finally displayed on the display screen 711 of the smartphone 1 will be described later.

### (Processing Procedure of Displaying Fake Level)

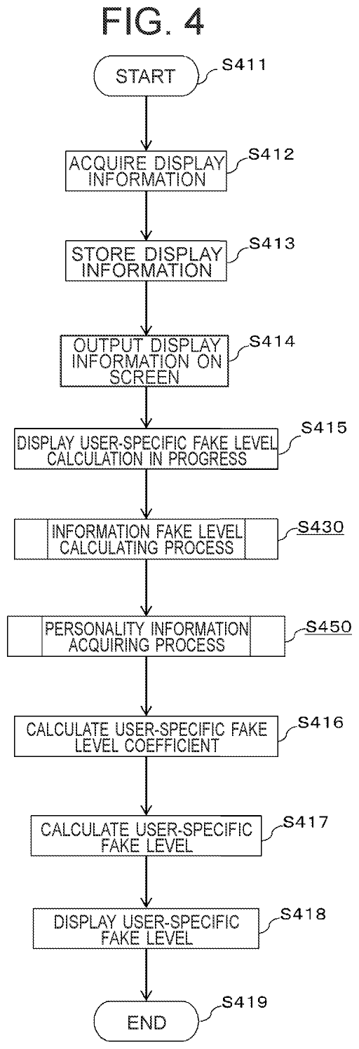

FIG. 4 illustrates a flowchart of a processing procedure of a fake level display process (corresponding to an information display method) according to the present embodiment. The processing procedure illustrated in FIG. 4 will be described with reference to the functional block diagram of FIG. 3.

Upon starting the fake level display process (step S411), the main processor 2 causes the display information acquiring section 13 to acquire display information to be displayed on the smartphone 1 (step S412).

Next, the main processor 2 causes the display information storing section 14 to store the display information acquired by the display information acquiring process (step S412) in the nonvolatile memory 42 of the storage 4 (step S413).

Next, the main processor 2 causes the display data output section 21 to output the display information stored by the display information storing process (step S413) to the display screen 711 of the smartphone 1 (step S414) for displaying the display information. The display information screen output process (step S414) described above is the same as a typical process carried out in the smartphone 1.

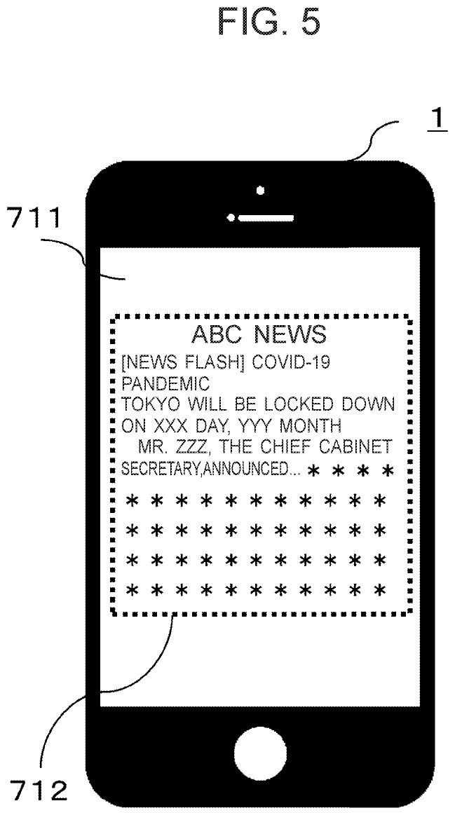

FIG. 5 illustrates a display example of the display information by the display information screen output process (step S414).

FIG. 5 illustrates the display screen 711 of the smartphone 1, in which the display information from the WEB page acquired through the internet 612 is being displayed in the display information area 712. Typically, the display information to be displayed in the display information area 712 of FIG. 5 is the same as the display information acquired by the user 10 who operates the smartphone 1.

Here, referring back to FIG. 4, the processing procedure of the fake level display process continues to be described.

After the display information screen output process (step S414), the main processor 2 carries out a process of showing that an information fake level relating to the display information is being calculated (user-specific fake level being calculated display process) (step S415).

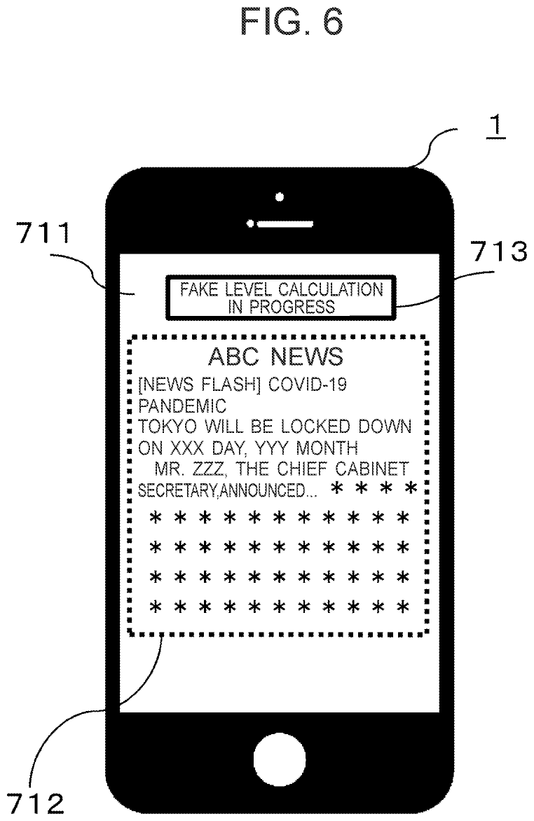

FIG. 6 illustrates a display example by the user-specific fake level being calculated display process (step S415).

FIG. 6 illustrates the fake level area 713 of the display screen 711, in which a sentence (character string “fake level calculation in progress”) expressing that a fake level is being calculated is displayed.

Displaying the sentence in the fake level area 713 enables the user 10 who operates the smartphone 1 to know that the information fake level relating to the display information is being calculated. In the present embodiment, two types of fake level including an information fake level specific to the content and a user-specific fake level for each user who has been in contact with the information are calculated, however, as illustrated in FIG. 6, without distinguishing these two types, an indication “fake level” may be simply displayed in the screen display example.

Here, referring back to FIG. 4, the processing procedure of the fake level display process continues to be described.

After the user-specific fake level being calculated display process (step S415), the main processor 2 carries out an information fake level calculating process (step S430) which is a predefined process (subroutine).

The information fake level calculating process (step S430), which is a subroutine, is a process of calculating an information fake level relating to display information, by which the information fake level relating to the display information can be obtained.

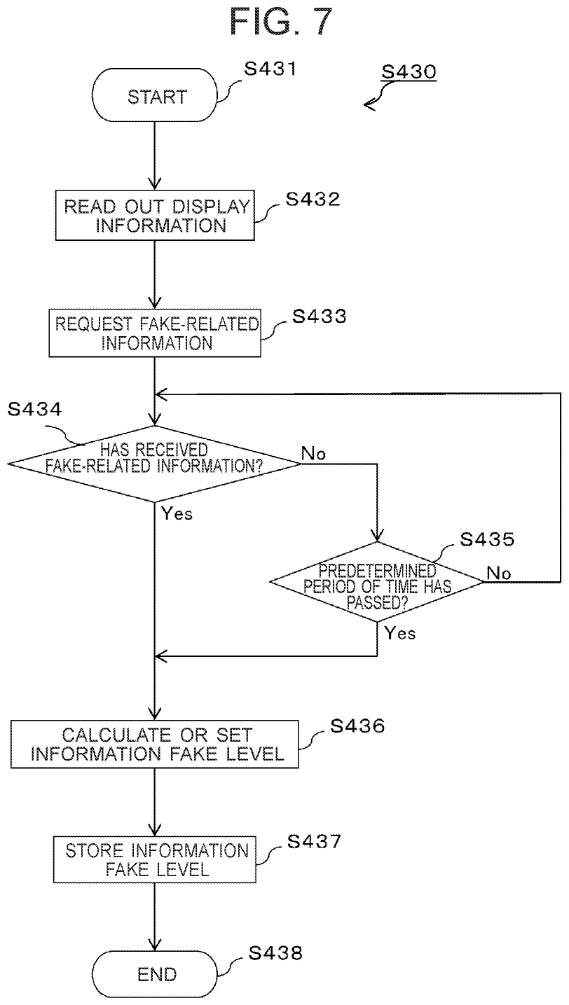

In the following, the information fake level calculating process (step S430), which is a subroutine, will be described. FIG. 7 illustrates a flowchart of a processing procedure of the information fake level calculating process (step S430) as a subroutine. The processing procedure of FIG. 7 will be described with reference to the functional block diagram of FIG. 3.

Upon starting the information fake level calculating process (step S430) (step S431), the main processor 2 reads out the display information stored by the display information storing section 14 and loads the display information into the RAM 43 (step S432). Of course, the display information acquiring section 13 may acquire the display information to be displayed, again.

Next, the main processor 2 requests the external server 615 that issues fake-related information to transmit fake-related information by means of the communication processor 12 (step S433).

Next, the main processor 2 determines whether the fake-related information has been received from the external server 615 that issues fake-related information (step S434).

In the fake-related information reception determining process (step S434), when determining that the fake-related information has not been received (step S434/No), the main processor 2 determines whether a predetermined period of time has passed (step S435). The “predetermined period of time” referred herein is a period of time provided to stand by until the fake-related information is received.

In the predetermined period of time passage determining process (step S435), when determining that the predetermined period of time has not passed (step S435/No), the main processor 2 returns to the fake-related information reception determining process (step S434). The predetermined period of time passage determining process (step S435) can prevent the information fake level calculating process (step S430) from being deadlocked.

On the other hand, in the predetermined period of time passage determining process (step S435), when determining that the predetermined period of time or more has passed (step S435/Yes), the main processor 2 proceeds to the information fake level calculating process (step S436).

The main processor 2 proceeds to the information fake level calculating process (step S436) when determining that, in the fake-related information reception determining process (step S434), the fake-related information has been received (step S434/Yes) or when the predetermined period of time or more has passed in step S435 (step S435/Yes).

In the information fake level calculating process in step S436, the main processor 2 calculates an information fake level by AI processing or a conventional rule-based calculation process (step S436).

In the information fake level calculating process (step S436), the main processor 2 comprehensively consider the fake-related information acquired from the external server 615 by the fake-related information acquiring section 15, past display information, supplementary information (past fake level, etc.) relating to the display information, and the like to apply inference and calculate an information fake level.

In this connection, there may be cases that information relating to a fake level is attached to the display information itself. For example, Twitter (Twitter, Inc., USA) labels the content of SNS (Tweets) to let the users to know that the tweets may contain misleading information or information of which credibility is questioned. In the information fake level calculating process (step S436), it is inferred that an information level of the SNS (tweet) thus labeled is high.

In the predetermined period of time passage determining process (step S435), when determining that the predetermined period of time has passed and thus proceeding to step S436 (information fake level calculating process), the main processor 2 uses past fake information to apply inference and calculate an information fake level. If no past fake information is available, the main processor 2 may set a predetermined fake level. A predetermined fake level to be set here is not specifically defined, however, considering that the fake-related information has not been acquired, it may be set to an intermediate level (fake level 50%).

Next, the main processor 2 causes the information fake level data storing section 17 to store the information fake level acquired by the information fake level calculating process (step S436) or the information fake level set by the information fake level setting process (step S436) in the nonvolatile memory 42 of the storage 4 (step S437).

Thus, the processing procedure of the information fake level calculating process (step S430) is completed (step S438).

Here, referring back to FIG. 4, the processing procedure of the fake level display process continues to be described.

After the information fake level calculating process (step S430), the main processor 2 proceeds to a personality information acquiring process (step S450), which is a predefined process (subroutine). The personality information acquiring process (step S450) is a process of acquiring personal information such as personality of the user 10 who operates the smartphone 1. In the following, the personality information acquiring process (step S450), which is a subroutine, will be described.

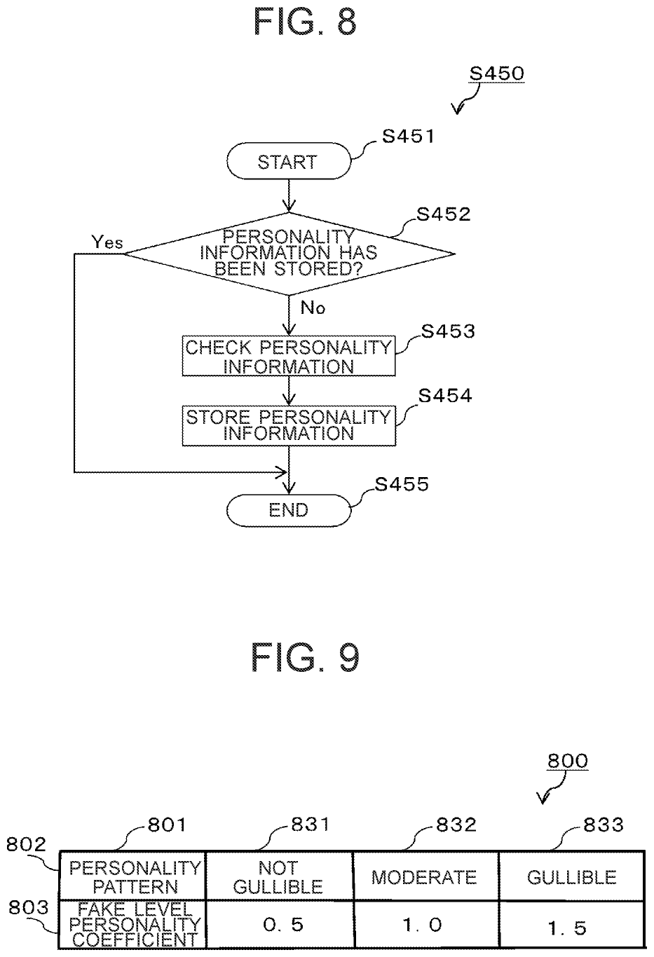

FIG. 8 illustrates a flowchart of a processing procedure of the personality information acquiring process (step S450). The processing procedure illustrated in FIG. 8 will be described with reference to the functional block diagram of FIG. 3. The personality information in FIG. 8 is about the personality of a user in terms of gullibility.

Upon starting the personality information acquiring process (step S450) (step S451), the main processor 2 determines whether user personality information has been already stored by the personality information storing section 19 (step S452).

When determining that the user personality information is already stored in the personality information storage determining process (step S452), since it means that the personality information has been already acquired (step S452/Yes), the main processor 2 terminates this personality information acquiring process (step S450) (step S455). Of course, this personality information may be acquired again.

When determining that the user personality information is not stored (step S452/No), the main processor 2 proceeds to a subsequent process which is a personality information checking process (step S453).

The personality information checking process (step S453) is a process in which the main processor 2 causes the personality information acquiring section 18 to check the personality of a user (gullibility). In this case, the personality information acquiring section 18 asks various questions and obtains the answers to these questions to check the personality of the user in terms of gullibility.

In order to check the gullibility, various methods and websites for personality analysis regarding the gullibility which are widely available in the world may be used. For example, the Japanese Consumer Affairs Agency releases a questionnaire with fifteen items, such as “are you easy to be flattered?”, accepts the answers thereto on a scale of 1 to 5, from “hardly ever” (1) to “very much” (5), and calculates, using the total scores, the probability that the answerer who is approached with any offer may make a contract based on a scale of 1 to 5 (approximately 25%, approximately 30%, approximately 40%, approximately 50%, and approximately 70%). In the present embodiment, for convenience's sake, a result of the gullibility check is classified into three patterns (not gullible, moderate, gullible).

Next, the main processor 2 causes the personality information storing section 19 to store the user personality information obtained by the personality information checking process (step S453) in the nonvolatile memory 42 of the storage 4 (step S454), and completes the personality information acquiring process (step S450) (step S455).

Here, referring back to FIG. 4, the processing procedure of the fake level display process continues to be described.

After the personality information acquiring process (step S450), the main processor 2 carries out a user-specific fake level coefficient calculating process (step S416).

The user-specific fake level coefficient calculating process (step S416) is a process of calculating a user-specific fake level coefficient (fake level personality coefficient) based on the user personality information (three patterns for gullibility) stored by the personality information storing process (step S454).

In the following, the user-specific fake level coefficient (fake level personality coefficient) based on the user personality information will be described.

FIG. 9 illustrates a table (800) in which the user-specific fake level coefficients (fake level personality coefficients) for gullibility (three categories) are listed.

In the items (column 801), an item of personality patterns (row 802) and an item of fake level personality coefficients (row 803) are included.

The item of personality patterns (row 802) includes three personality patterns for gullibility (not gullible (column 831), moderate (column 832), and gullible (column 833)). The item of fake level personality coefficients (row 802) includes user-specific fake level personality coefficients (fake level personality coefficients) corresponding to the three patterns in the personality patterns (row 802), respectively. According to this table, for example, a user-specific fake level coefficient (fake level personality coefficient) of a user whose user personality pattern falling within “moderate” for gullibility is 1.0.

Here, referring back to FIG. 4, the processing procedure of the fake level display process continues to be described.

After the user-specific fake level coefficient calculating process (step S416), the main processor 2 carries out a user-specific fake level calculating process (step S417).

The user-specific fake level is a fake level to be finally displayed in the fake level area 713 of the smartphone 1, in which the user personality information (gullibility) is reflected.

The user-specific fake level is calculated, as expressed in the following Equation (1), using the product of an information fake level and a fake level personality coefficient.

Yₙ=x_{A}*w₁ₙ (1)

where,

Yₙ: user-specific fake level of user-n for information-A,

x_{A}: information fake level of information-A, and

w₁ₙ: fake level personality coefficient of user-n (user-specific fake level coefficient).

For example, in the case where the information fake level is 50% and the personality pattern (gullibility) falls within moderate, the user-specific fake level is the product of the information fake level (50%) and the fake level personality coefficient (1.0), which is 50% (50%×1.0). In the case where the information fake level is 50% and the personality pattern (gullibility) falls within “gullible”, the user-specific fake level is the product of the information fake level (50%) and the fake level personality coefficient (1.5), which is 75% (50%×1.5).

In the present embodiment, in all cases where a user-specific fake level, which is the product of an information fake level and a fake level personality coefficient, exceeds 100%, the user-specific fake level is set to 100% of the maximum value. However, fake level 100% does not mean that the information is completely fake objectively (=credibility is 0), but means that it is determined that the fake level thereof is the maximum value in terms of alerting an individual user.

Next, the user-specific fake level calculated in the user-specific fake level calculating process (step S417) is displayed in the fake level area 713 of the smartphone 1 (step S418), and the processing procedure of the fake level displaying process in FIG. 4 is completed (step S419).

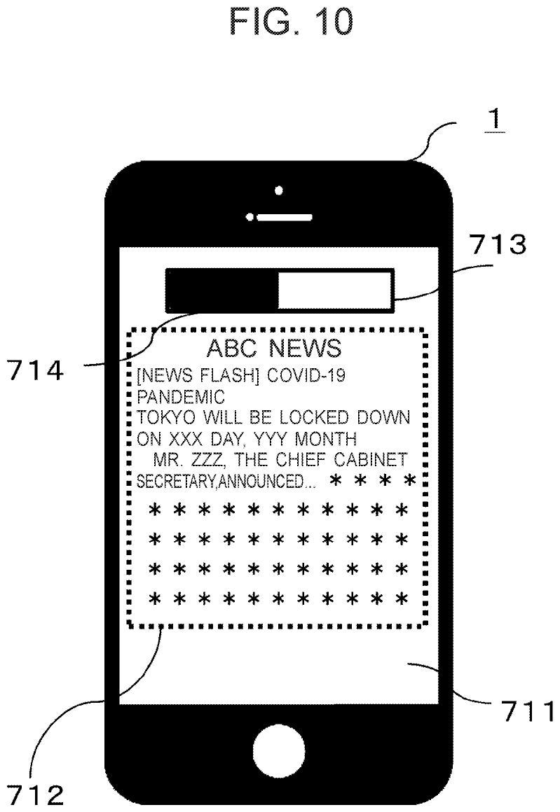

FIG. 10 illustrates a display example of the process (user-specific fake level displaying process) in step S418.

In FIG. 10, a user-specific fake level 714 is displayed in the fake level area 713 of the display screen 711 using a bar graph. The display example in FIG. 10 is an example that the user-specific fake level is about 50%.

Displaying the bar graph (about 50%) in the fake level area 713 allows the user 10 who operates the smartphone 1 to know the user-specific fake level.

In FIG. 10, the fake level (user-specific fake level) of the whole display information in the display information area 712 is displayed, however, only a target sentence of which the fake level (user-specific fake level) has been determined may be displayed by highlighting, blinking, or the like, in order to make it distinguishable.

In the present embodiment, a user-specific fake level is displayed using a bar graph, however, the user-specific fake level can be expressed using a numerical value and displayed as text information (for example, a character string “50%”).

In case that the fake level is high, for calling attention, a warning may be newly displayed on the display screen 711 or a warning sound may be output.

In displaying a warning, considering that fake information tends to spread within a short period of time, in case that the information fake level of the acquired information is high, specifically, when the user-specific fake level is equal to or more than a predetermined first warning threshold value, the warning may be displayed triggered by the attempt of the user to forward or spread the information. The warning in this case is, for example, “This information is most likely a fake. Do you really want to forward it?”. Alternatively, for example, when the information fake level is equal to or more than the first warning threshold value even if the information fake level is less than the first warning threshold value, another warning in which the degree of warning is lower than that of the warning mentioned above, which is, for example, “This information is possibly a fake. Do you really want to forward it?” may be displayed.

In the present embodiment, a fake level to be displayed (user-specific fake level) is displayed using a bar graph, however, the fake level may be classified into roughly three stages (for example, three stages such as “likely reliable”, “need attention”, and “likely unreliable”) and displayed. In this case, since the fake level (user-specific fake level) is identified on a scale of three stages, various approaches may be employed.

For example, providing the display information area or the target sentence with an enclosing line realizes identification by means of the enclosing line (thickness, type (dotted line, solid line, broken line, or the like), color, or the like of the enclosing line). Alternatively, providing the display information area or the target sentence with a background realizes identification by means of the background (color of the background, type of the texture pattern, and the like).

In the present embodiment, the display information (display information from WEB pages and display information from SNS) acquired through networks is obtained from the external server 615 through the networks to calculate a fake level (information fake level) thereof. On the other hand, inference and calculation of a fake level (information fake level) can also be carried out by comprehensively considering past display information, supplementary information about the display information (past fake level, etc.) using the AI processing inside the smartphone 1 without depending on the external server 615.

According to the present embodiment, in displaying a fake level relating to display information, incorporating personality information (gullibility) about a user who handles the display information into a fake level (information fake level) thereof allows the user to be visually provided with a fake level (user-specific fake level) optimal for the user who is viewing the display information.

Note that, in the present embodiment, as illustrated in FIG. 9, personality of the user is classified into three patterns (three patterns of “not gullible”, “moderate”, and “gullible”), however, the number of patterns is not limited to three, and may be increased or decreased.

Furthermore, in the present embodiment, a user-specific fake level is calculated by multiplication using the fake level personality coefficients illustrated in FIG. 9, however, multiplication operation does not necessarily have to be used, but weighting with statistical operations or the like may be used.

### Second Embodiment

Hereinafter, a second embodiment of the present invention will be described. Note that the basic hardware configuration of the second embodiment is the same as that of the first embodiment described above, and in the following, the differences between the present embodiment (second embodiment) and the embodiment described above (first embodiment) will be mainly described, and repetitive explanation for the common components will be omitted as much as possible to avoid duplication.

In the embodiment described above, the user personality information in terms of gullibility is used as information about a user. On the other hand, in the present embodiment, physical condition information about a user is used as the information about the user.

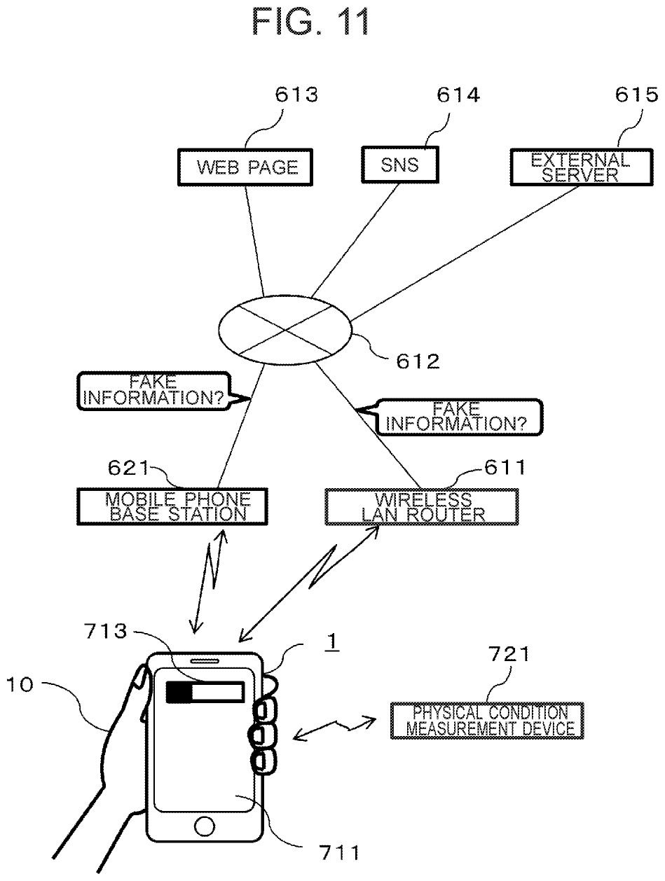

FIG. 11 is a schematic diagram for explaining an outline of the present embodiment (second embodiment).

FIG. 11 is almost the same as the schematic diagram of FIG. 1, however, additionally includes a physical condition measurement device 721 connected to the smartphone 1 by near field wireless communication. The physical condition measurement device 721 is a device for measuring physical conditions of the user 10, and is configured separately from the smartphone 1, which thus corresponds to an external physical condition measurement device.

It is generally known that a person who is in a state of excitement tends to be unable to make a sound judgment. In the present embodiment, a state of excitement of a user is estimated based on physical condition information acquired from the physical condition measurement device 721 so as to reflect a level of excitement in a fake level and display it.

### (Functional Block According to Present Embodiment)

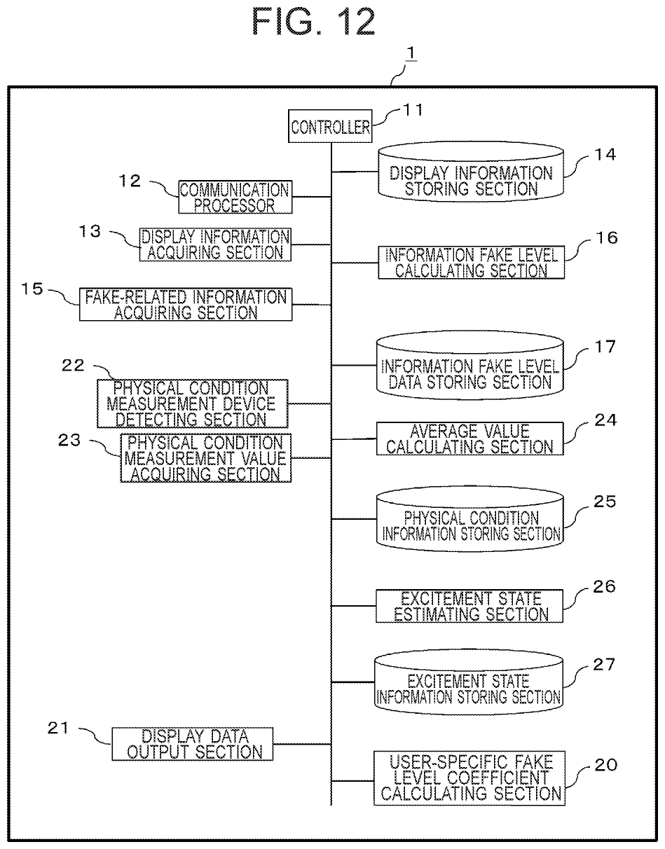

FIG. 12 is a functional block diagram illustrating an example of a functional block configuration of the present embodiment.

The functional block diagram of FIG. 12 includes the functions that overlap with those of the functional block diagram (FIG. 3) of the first embodiment, and repetitive explanation for these functions will be omitted. In the following, only the functions which are newly added in the present embodiment and the functions which need to be additionally explained will be described.

The communication processor 12 in the present embodiment is a function capable of transmitting and receiving information to and from an external device capable of near field wireless communication by means of the near field wireless communication I/F 63 (see FIG. 2). In the present embodiment, the physical condition information is mainly received from the physical condition measurement device 721.

In recent years, the physical condition measurement devices 721 capable of near field wireless communication such as Bluetooth (registered trademark) communication or NFC standard communication have been widely used, by which various kinds of physical condition information (blood pressure, body temperature, respiration rate, heart rate, sweat rate, and the like) can be acquired. In the present embodiment, the physical condition measurement devices 721 capable of near field wireless communication descried above are used. In many cases, the physical condition measurement devices 721 of these types are referred to as wearable devices, and built in devices such as watch type devices, wristband type devices, or devices to be attached to the body.

A physical condition measurement device detecting section 22 is a function of searching for the physical condition measurement devices 721 which are in communicable states by means of the near field wireless communication I/F 63. Using the physical condition measurement device detecting section 22 enables grasping of the type and number of communicable physical condition measurement devices 721.

A physical condition measurement value acquiring section 23 is a function of acquiring physical condition measurement values from the physical condition measurement devices 721 detected by the physical condition measurement device detecting section 22.

An average value calculating section 24 is a function of calculating an average value of the physical condition measurement values. Values of various pieces of physical condition information described above differ among individuals, and thus a level of excitement cannot be determined by comparing these values with an absolute numerical value. In this regard, providing the average value calculating section 24 enables comparison of a physical condition measurement value acquired by the physical condition measurement value acquiring section 23 with a daily physical condition measurement value, and thus determination as to how much the physical condition measurement value has increased or decreased based on the comparison result. Calculating an average value means calculation of so-called physical condition information under normal conditions.

In the present embodiment, an average (moving average) of physical condition measurement values for two weeks (14 days) is obtained, and the value thus obtained is set as an average value.

The timing of measuring physical conditions can be arbitrarily chosen, for example, every day at a fixed time, at a fixed time interval, at all times, and the like.

In case that not all the physical condition measurement values of two weeks are available, a physical condition measurement value for the shortage is supplied for calculation of an average value. In the present embodiment, a statistical standard value (same sex, same age, and approximately same body size) is adopted as a physical condition measurement value to be compensated.

A physical condition information storing section 25 is a function of storing, in the nonvolatile memory 42 of the storage 4, the physical condition measurement value acquired by the physical condition measurement value acquiring section 23 and the average value up to the previous day calculated by the average value calculating section 24.

An excitement state estimating section 26 is a function of comparing the physical condition measurement value acquired by the physical condition measurement value acquiring section 23 with the average value of the physical condition measurement values up to the previous day calculated by the average value calculating section 24, determining how much the physical condition measurement value has increased or decreased, and estimating a state of excitement based on the result. In the present embodiment, a state of excitement is classified into three stages (three stages of “normal”, “slightly excited”, and “excited”). An excitement state information storing section 27 is a function of storing, in the nonvolatile memory 42 of the storage 4, excitement state information indicating a state of excitement (three stages) estimated by the excitement state estimating section 26.

The user-specific fake level coefficient calculating section 20 is a function of calculating a coefficient (weighting) for the information fake level obtained by the information fake level calculating section 16 using the user excitement state information stored by the excitement state information storing section 27. A fake level to be displayed is determined by this user-specific fake level coefficient calculating section 20.

A coefficient (weighting) for a fake level (information fake level) will be described later.

The display data output section 21 is a function of displaying, on the display screen 711 of the smartphone 1, display information stored in the nonvolatile memory 42 of the storage 4 by the display information storing section 14, a user-specific fake level in which the user physical condition information is incorporated into the information fake level relating to the display information, and the like.

### (Processing Procedure of Displaying Fake Level According to Present Embodiment)

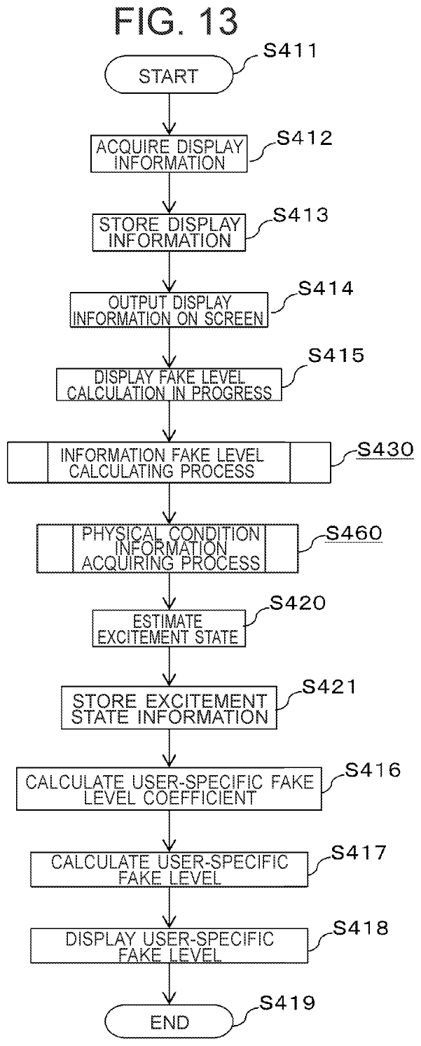

FIG. 13 illustrates a flowchart of a processing procedure of a fake level display process according to the present embodiment.

The flowchart in FIG. 13 includes the processes that overlap with those of the flowchart of the first embodiment (FIG. 4), and repetitive explanation therefor will be omitted. In the following, only the processes which are newly added in the present embodiment and the processes which need to be additionally explained will be described.

The processing procedure of FIG. 13 will be described with reference to the functional block diagram of FIG. 12.

In the flowchart (FIG. 13) of displaying a fake level in the present embodiment, instead of the personality information acquiring process (step S450) of the first embodiment described above, several processes (physical condition information acquiring process (step S460), excitement state estimating process (step S420), and excitement state storing process (step S421)) are added.

In the following, the physical condition information acquiring process (step S460), which is a subroutine, will be described.

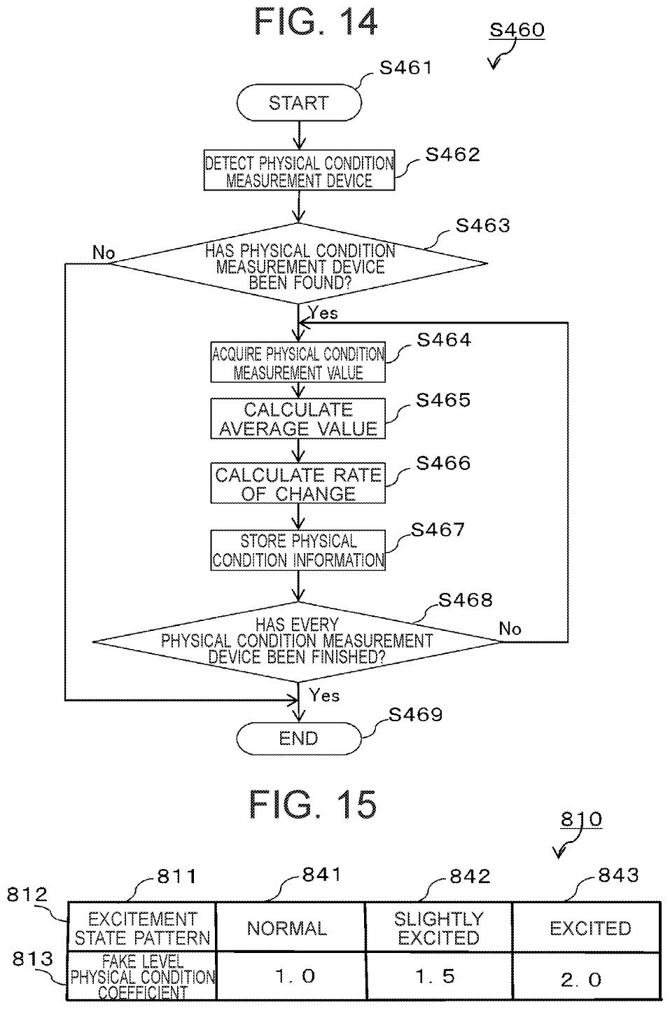

FIG. 14 illustrates a flowchart of a processing procedure of the physical condition information acquiring process (step S460).

The processing procedure of FIG. 14 will be described with reference to the functional block diagram of FIG. 12.

Upon start of the physical condition information acquiring process (step S460) (step S461), the physical condition measurement device detecting section 22 detects the physical condition measurement devices 721 which are in communicable states (step S462).

Next, it is determined whether the physical condition measurement devices 721 have been detected (step S463).

If it is determined in the physical condition measurement device found/unfound determining process (step S463) that no physical condition measurement device 721 capable of being connected is found (step S463/No), since this means that the physical condition measurement value cannot be acquired, the physical condition information acquiring process (step S460) is terminated (step S469).

In the physical condition measurement device found/unfound determining process (step S463), when it is determined that the physical condition measurement device 721 capable of being connected is found (step S463/Yes), the physical condition measurement value acquiring section 23 acquires a physical condition measurement value from the physical condition measurement device 721 which has been connected (step S464).

Next, the average value calculating section 24 calculates an average value of the physical condition measurement values up to the previous day stored by the physical condition information storing section 25 (step S465).

Various methods for calculating an average value are adaptable, for example, calculating an average value of physical condition measurement values within the latest predetermined number of days, an average value of physical condition measurement values in the latest predetermined number of units, and the like. In the present embodiment, an average value of physical condition measurement values in fourteen days up to the previous day is calculated and used.

In the present embodiment, if not all the physical condition measurement values in the latest fourteen days are available, a physical condition measurement value for the shortage is compensated therefor to calculate an average value of the physical condition measurement values.

Next, how much the physical condition measurement value acquired in the physical condition measurement value acquiring process (step S464) has changed with respect to the average value of the physical condition measurement values calculated in the average value calculating process (step S465) is calculated as a rate of change from the average value (step S466).

Next, the physical condition information storing section 25 stores, in the nonvolatile memory 42 of the storage 4 (step S467), the physical condition measurement value currently acquired in the physical condition measurement value acquiring process (step S464) and the change rate of the physical condition measurement value calculated in the change rate calculating process (step S466).

Next, it is determined whether, regarding every physical condition measurement device 721 detected in the physical condition measurement device detecting process (step S462), all the physical condition measurement values have been acquired (step S468).

When it is determined in the all-physical condition measurement device completion determining process (step S468) that not all the physical condition measurement devices 721 have been finished, (step S468/No), the unconnected physical condition measurement device 721 is caused to be connected and then the processing proceeds to the physical condition measurement value acquiring process (step S464).

When it is determined in the all-physical condition measurement device completion determining process (step S468) that all the physical condition measurement devices 721 have been finished (step S468/Yes), since there is no unconnected physical condition measurement device 721, the physical condition information acquiring process (step S460) is completed (step S469).

Here, referring back to FIG. 13, the processing procedure of the fake level display process continues to be described.

After the physical condition information acquiring process (step S460), the excitement state estimating process (step S420) is carried out by the excitement state estimating section 26.

The degree of affecting a state of excitement varies depending on the measurement items of the physical condition measurement. In the present embodiment, the weighting is carried out for the measurement items of the physical condition measurement by the AI processing.

Various estimation methods for estimating a state of excitement are adaptable, for example, classifying a state of excitement using a range of the physical condition measurement values, classifying it using a rate of change of a physical condition measurement value with respect to an average value, and the like. In the present embodiment, a method of classifying a state of excitement using a rate of change of a physical condition measurement value with respect to an average value is employed.

In the following, an example where the physical condition measurement value is a respiratory rate will be described. In the present embodiment, it is predetermined that a physical condition measurement value that is less than +10% of an average value is a normal state, a physical condition measurement value that is more than or equal to +10% of an average value to less than +30% is a slightly excited state, and a physical condition measurement value that is more than or equal to +30% of an average value is an excited state. For example, where the average value of respiratory rate is 15 breaths per minute, the physical condition measurement value that is less than 16.5 times per minute is classified as the normal state, the physical condition measurement value that is more than or equal to 16.5 breaths per minute and less than 19.5 breaths per minute classified as the slightly excited state, and the physical condition measurement value that is more than or equal to 19.5 breaths per minute is classified as the excited state. Of course, the object of the present embodiment can be achieved by classification methods other than the one using a rate of change. For example, a method of classifying a state of excitement based on a difference value between an average value and a physical condition measurement value, a method of classifying it considering a variation and a rate of variation in elements used for calculating an average value, and the like are adaptable.

Next, the excitement state information storing section 27 stores the excitement state information based on the result estimated in the excitement state estimating process (step S420) in the nonvolatile memory 42 of the storage 4 (step S421).

Next, the processing proceeds to the user-specific fake level coefficient calculating process (step S416).

The user-specific fake level coefficient calculating process (step S416) is a process of calculating a user-specific fake level coefficient (fake level physical condition coefficient) based on a state of excitement of the user (three categories) stored by the excitement state storing process (step S421).

In the following, the user-specific fake level coefficient (fake level physical condition coefficient) based on the physical condition information will be described.

FIG. 15 illustrates a table (810) in which the user-specific fake level coefficients (fake level physical condition coefficient) for each state of excitement (three patterns) are listed.

In the item (column 811), an item of excitement state patterns (row 812) and an item of fake level physical condition coefficients (row 813) are included.

The item of excitement state patterns (row 812) includes three patterns for a state of excitement (normal state (column 841), slightly excited state (column 842), and excited state (column 843)). The item of fake level physical condition coefficients (row 813) includes user-specific fake level physical condition coefficients (fake level physical condition coefficients) corresponding to the three patterns of the state of excitement, respectively (row 812).

For example, a state of excitement of the user falling within “normal” means that the user-specific fake level coefficient (fake level physical condition coefficient) is 1.0.

Here, referring back to FIG. 13, the processing procedure of the fake level display process continues to be described.

After the user-specific fake level coefficient calculating process (step S416), the user-specific fake level calculating process (step S417) is carried out.

The user-specific fake level is a fake level to be finally displayed in the fake level area 713 of the smartphone 1, in which the physical condition information (state of excitement) is reflected.

The user-specific fake level is calculated, as expressed in the following Equation (2), using the product of an information fake level and a fake level physical condition coefficient.

Yₙ=x_{A}*w₂ₙ (2)

where,

Yₙ: user-specific fake level of user-n for information-A,

x_{A}: information fake level of information-A, and

w₂ₙ: fake level physical condition coefficient of user-n (user-specific fake level coefficient).

For example, in the case where the information fake level is 50% and the state of excitement corresponds to the normal state, the user-specific fake level is the product of the information fake level (50%) and the fake level physical condition coefficient (1.0), which is 50% (50%×1.0). In the case where the information fake level is 50% and the state of excitement corresponds to the slightly excited state, the user-specific fake level is the product of the information fake level (50%) and the fake level physical condition coefficient (1.5), which is 75% (50%×1.5).

In the present embodiment as well, in all cases where a user-specific fake level, which is the product of an information fake level and a fake level physical condition coefficient, exceeds 100%, the user-specific fake level is set to 100% of the maximum value.

Next, the user-specific fake level calculated in the user-specific fake level calculating process (step S417) is displayed in the fake level area 713 of the smartphone 1 (step S418), and the processing procedure of the fake level displaying process in FIG. 13 is completed (step S419).

In the present embodiment, as illustrated in FIG. 15, a state of excitement of a user is classified into three patterns (three patterns of “normal”, “slightly excited”, and “excited”), however, the number of patterns is not limited to three, and may be increased or decreased.

Furthermore, in the present embodiment, a user-specific fake level is calculated by multiplication using the fake level physical condition coefficients illustrated in FIG. 15, however, multiplication operation does not necessarily have to be used, but weighting with statistical operations or the like may be used.

In the present embodiment, a respiration rate is used for explanation as physical condition information. In this regard, a state of excitement may be detected using the blood pressure, body temperature, heart rate, sweat rate, and the like as physical condition information, and moreover, combining a plurality of pieces of the physical condition information enables more accurate detection of a state of excitement.

In the description above, the external physical condition measurement device 721 carries out the physical condition measurement of a user. In this regard, if the smartphone 1 can solely carry out the physical condition measurement, measurement values acquired thereby can be used.

For example, irradiating capillaries of a user's fingertip with a light-emitting diode (LED) light of a smartphone and analyzing an image of the capillaries of the user's fingertip with a function of a camera of the smartphone enables measurement of the pulse rate, blood pressure, and the like.

According to the present embodiment, in displaying a fake level relating to display information, incorporating physical condition information (state of excitement) about a user who handles the display information into a fake level thereof allows the user to be visually provided with a fake level (user-specific fake level) optimal for the user who is viewing the display information.

### Third Embodiment

Hereinafter, a third embodiment of the present invention will be described. Note that the basic hardware configuration of the third embodiment is the same as those of the embodiments described above, and in the following, the differences between the present embodiment (third embodiment) and the embodiments described above will be mainly described, and repetitive explanation for the common components will be omitted as much as possible to avoid duplication.

In the first embodiment described above, user personality information (gullibility) is used as information about a user. Furthermore, in the second embodiment described above, user physical condition information (state of excitement) is used as information about a user.

In the present embodiment, both the user personality information (gullibility) and the user physical condition information (state of excitement) are reflected in a fake level and displayed as information about a user.

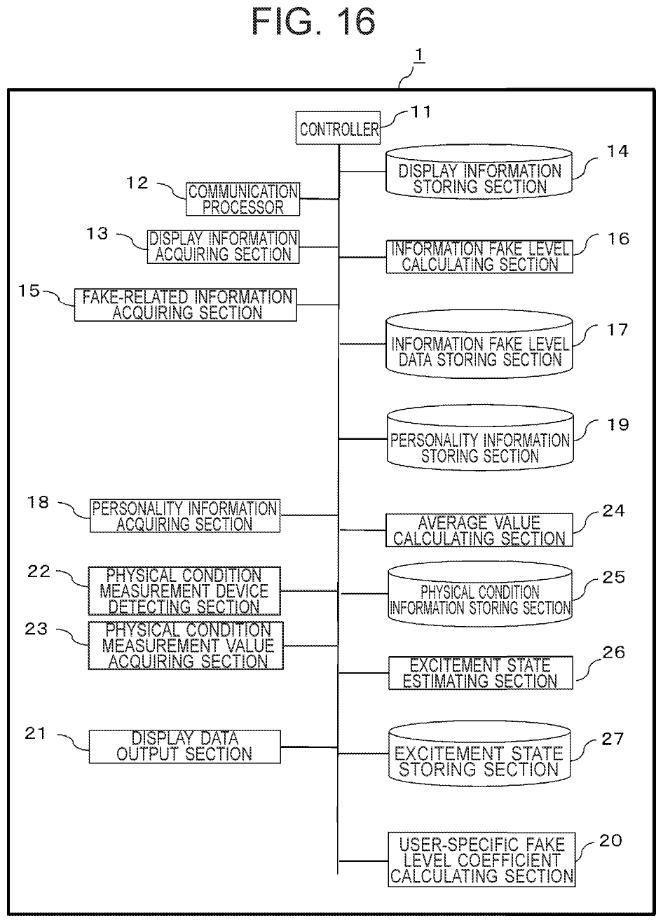

FIG. 16 is a functional block diagram illustrating an example of a functional block configuration of the present embodiment.

The functional block diagram of FIG. 16 includes the functions that overlap with those of the functional block diagram (FIG. 3) of the first embodiment, and also includes the functions that overlap with those of the functional block diagram (FIG. 12) of the second embodiment. Accordingly, repetitive explanation for these functions will be omitted. In the following, only the functions which need to be additionally explained will be described.

The user-specific fake level coefficient calculating section 20 is a function of calculating a coefficient (weighting) for an information fake level obtained by the information fake level calculating section 16 using the user personality information stored by the personality information storing section 19 and the user excitement state information stored by the excitement state information storing section 27. The fake level to be displayed is determined by this user-specific fake level coefficient calculating section 20.

A coefficient (weighting) for an information fake level will be described later.

### (Processing Procedure of Displaying Fake Level According to Present Embodiment)

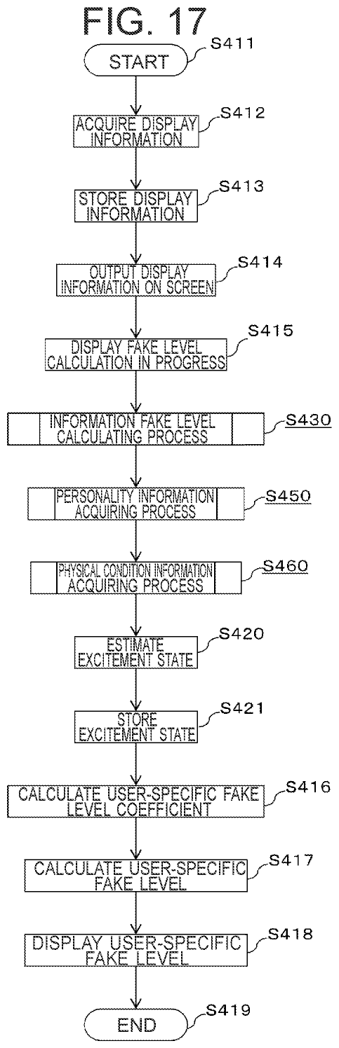

FIG. 17 illustrates a flowchart of a processing procedure of a fake level display process according to the present embodiment.

The flowchart in FIG. 17 includes the processes that overlap with those of the flowchart of the first embodiment (FIG. 4), and also the processes that overlap with those of the flowchart of the second embodiment (FIG. 13). Accordingly, repetitive explanation therefor will be omitted. In the following, only the processes which need to be additionally explained will be described.

The user-specific fake level coefficient calculating process (step S416) is a process of calculating a user-specific fake level coefficient based on user personality information (three patterns of gullibility) stored by the personality information storing process (step S454) and a state of excitement of a user (three patterns) stored by the excitement state storing process (step S421).

In the following, the user-specific fake level coefficient based on personality information (three patterns of gullibility) and physical condition information (three patterns of a state of excitement) will be described.

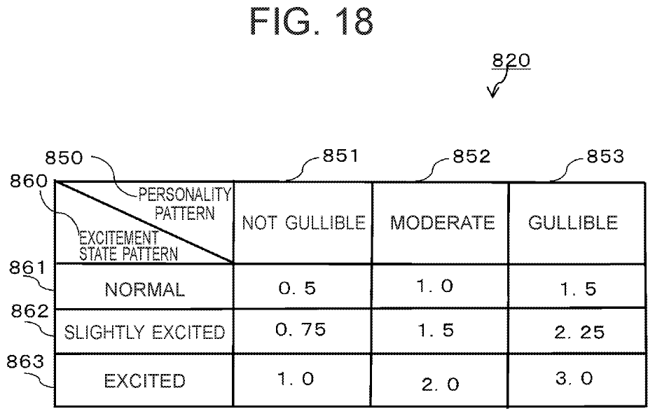

FIG. 18 is a table (820) in which the user-specific fake level coefficients for the three patterns of gullibility and the three patterns of a state of excitement are listed.

In the item, an item of personality patterns (row 850) and excitement state patterns (column 860) are included.

The personality patterns (row 850) are classified into three patterns in terms of gullibility (not gullible (column 851), moderate (column 852), and gullible (column 853)), and the excitement state patterns (column 860) are classified into three patterns in terms of a state of excitement (normal state (row 861), slightly excited state (row 862), and excited state (row 863)).

A user-specific fake level coefficient, which takes into consideration both user personality information (gullibility) and user physical condition information (state of excitement), is calculated, as expressed in the following Equation (3), using the product of a fake level personality coefficient and a fake level excitement coefficient.

w₃ₙ=w₁ₙ*w₂ₙ (3)

w₃ₙ: user-specific fake level coefficient

w₁ₙ: fake level personality coefficient of user-n

w2n: fake level excitement coefficient of user-n

For example, a case that gullibility is normal (fake level personality coefficient=1.0) and a state of excitement is normal (fake level physical condition coefficient=1.0) means that a user-specific fake level coefficient is 1.0 (product of fake level personality coefficient=1.0 and fake level physical condition coefficient=1.0).

The main processor 2 carries out the user-specific fake level calculating process (step S417) after the user-specific fake level coefficient calculating process (step S416).

The user-specific fake level is a fake level to be finally displayed in the fake level area 713 of the smartphone 1, in which both the user personality information (gullibility) and the user physical condition information (state of excitement) are reflected.

A user-specific fake level is calculated, as illustrated in the following Equation (4), using the product of an information fake level and a user-specific fake level coefficient.

yₙ=x_{A}*w₃ₙ=x_{A}*w₁ₙ*w₂ₙ (4)

where,

yₙ: user-specific fake level of user-n for information-A

x_{A}: information fake level of information-A

w₁ₙ: fake level personality coefficient of user-n

w₂ₙ: fake level physical condition coefficient of user-n

w₃ₙ: user-specific fake level coefficient

For example, in the case where an information fake level is 50% and a user-specific fake level coefficient is 1.0, a user-specific fake level is the product of the information fake level (50%) and the user-specific fake level coefficient (1.0), which is 50% (50%×1.0).

In the present embodiment as well, in all cases where a user-specific fake level, which is the product of an information fake level and a fake level physical condition coefficient, exceeds 100%, the user-specific fake level is set to 100% of the maximum value.

Next, the user-specific fake level calculated by the user-specific fake level calculating process (step S417) is displayed in the fake level area 713 of the smartphone 1 (step S418), and the processing procedure of the fake level displaying process in FIG. 17 is completed (step S419).

In the present embodiment, as illustrated in FIG. 18, personality of a user is classified into three patterns (three patterns of “not gullible”, “moderate”, and “gullible”), however, the number of patterns is not limited to three, and may be increased or decreased. Furthermore, a state of excitement of a user is classified into three patterns (three patterns of “normal”, “slightly excited”, and “excited”), however, the number of patterns is not limited to three, and may be increased or decreased.

Still further, in the present embodiment, a user-specific fake level is calculated by multiplication using the fake level physical condition coefficients illustrated in FIG. 18, however, multiplication operation does not necessarily have to be used, but weighting with statistical operations or the like may be used.

According to the present embodiment, in displaying a fake level relating to display information, incorporating both personality information (gullibility) and physical condition information (state of excitement) about a user who handles the display information into a fake level thereof allows the user to be visually provided with a fake level (user-specific fake level) optimal for the user who is viewing the display information.

### Fourth Embodiment

Hereinafter, a fourth embodiment of the present invention will be described. Note that the basic hardware configuration of the fourth embodiment is the same as those of the embodiments described above, and in the following, the differences between the present embodiment (fourth embodiment) and the embodiments described above will be mainly described, and repetitive explanation for the common components will be omitted as much as possible to avoid duplication.

The embodiments described above is provided for display information acquired through the internet, on the other hand, the present embodiment is provided for content of received broadcast. In the present embodiment, an information fake level is calculated based on characteristics relating to the credibility on audio information. Specifically, characteristics such as at least one or any combination of a broadcasting station that broadcasted the audio information, evaluation information in which the credibility of the audio information has been evaluated and the evaluation is provided, when the audio information was spread through public networks, how fast the audio information was spread through public networks, and sentence expressions included in the audio information may be used.

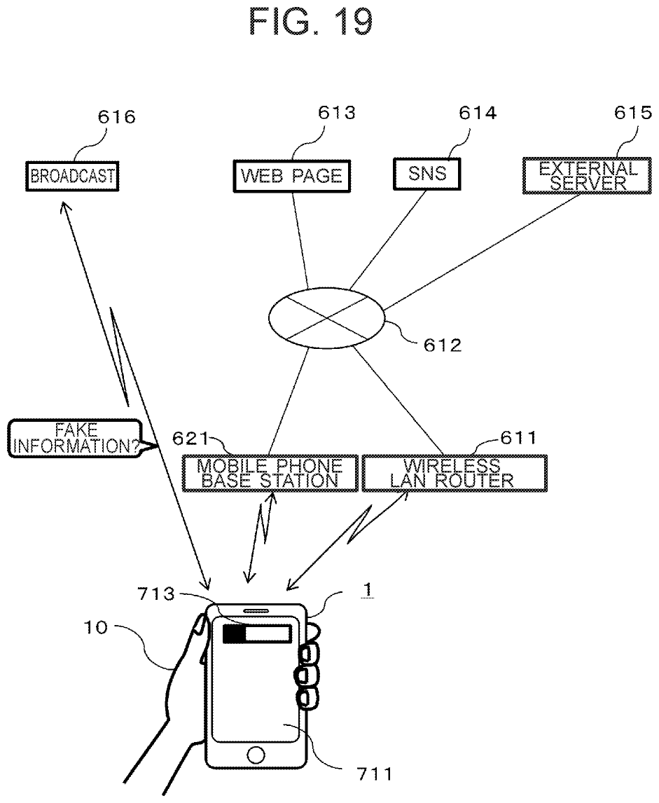

FIG. 19 is a schematic diagram for explaining an outline of the present embodiment (fourth embodiment).

FIG. 19 is a schematic diagram illustrating that the user 10 who is operating a mobile information terminal (smartphone 1) is acquiring, through the speaker 82 of the smartphone 1, a broadcast signal 616 from a television, radio, or the like. Video information is, of course, being displayed on the display screen 711 of the smartphone 1.

There is a possibility that audio information with doubtful credibility is mixed among the pieces of the audio information acquired by the user 10 who operates the smartphone 1 to receive the broadcast (FIG. 19 illustrates it as “fake information?”).

In the present embodiment, converting audio information into character information (text conversion) to make it equivalent to display information according the embodiments described above enables calculation of a fake level.

### (Functional Block According to Present Embodiment)

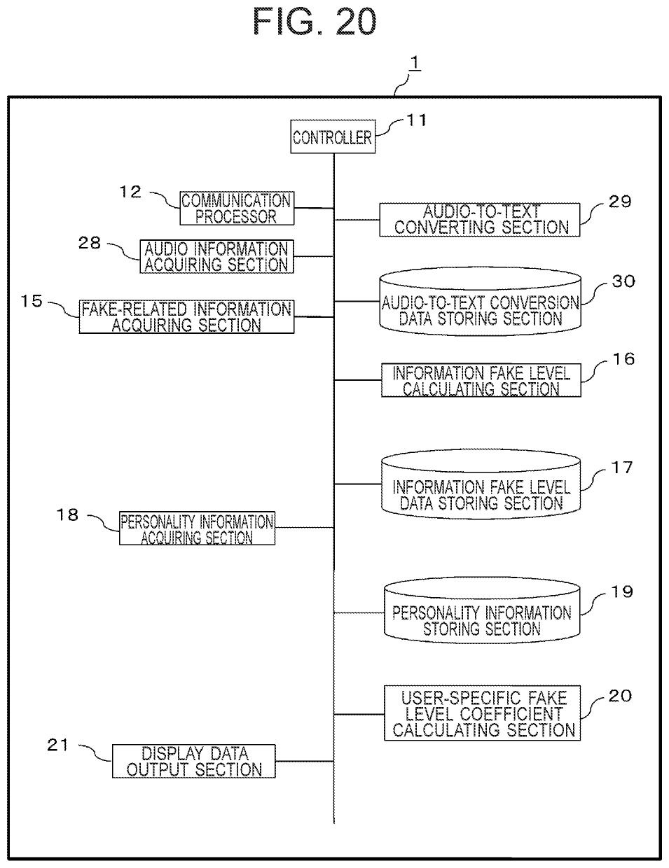

FIG. 20 is a functional block diagram illustrating an example of a functional block configuration according to the present embodiment.

The functional block diagram of FIG. 20 includes the functions that overlap with those of the functional block diagram (FIG. 3) of the first embodiment, and repetitive explanation for these functions will be omitted. In the following, only the functions which are newly added in the present embodiment and the functions which need to be additionally explained will be described.

The communication processor 12 in the present embodiment is a function of receiving a broadcast from a television, radio, or the like by means of the broadcast receiver 64.

An audio information acquiring section 28 is a function of acquiring audio information of a broadcast received by the communication processor 12.

An audio-to-text converting section 29 is a function of converting the audio information acquired by the audio information acquiring section 28 into character information (text conversion).

An audio-to-text conversion data storing section 30 is a function of storing the character information converted by the audio-to-text converting section 29 in the nonvolatile memory 42 or various RAMs 43 of the storage 4.

The display data output section 21 is a function of displaying, on the display screen 711 of the smartphone 1, the character information stored by the audio-to-text conversion data storing section 30, a fake level in which user personality information is incorporated to an information fake level relating to the character information, and the like.

### (Processing Procedure of Displaying Fake Level According to Present Embodiment)

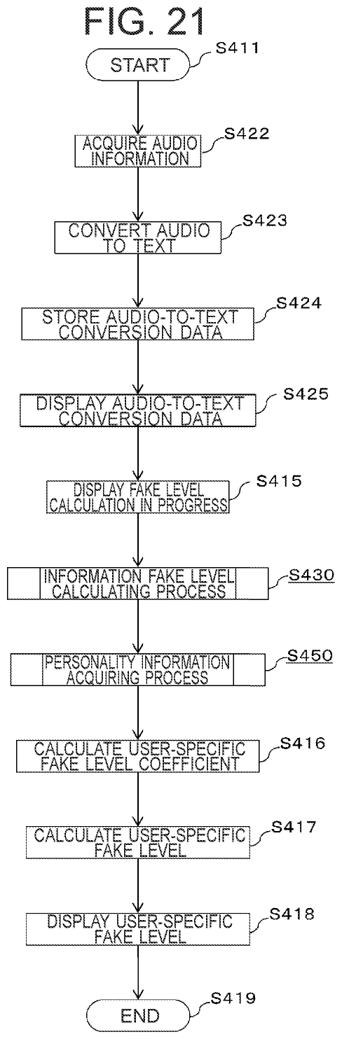

FIG. 21 illustrates a flowchart of a processing procedure of the fake level display process according to the present embodiment.

The flowchart in FIG. 21 includes the processes that overlap with those of the flowchart of the first embodiment (FIG. 4), and repetitive explanation therefor will be omitted. In the following, only the processes which are newly added in the present embodiment and the processes which need to be additionally explained will be described.

The processing procedure of FIG. 21 will be described with reference to the functional block diagram of FIG. 20.

In the flowchart (FIG. 21) of displaying a fake level according to the present embodiment, instead of some processes of the first embodiment described above, four processes (audio information acquiring process (step S422), audio-to-text converting process (step S423), audio-to-text conversion data storing process (step S424), and audio-to-text conversion data displaying process (step S425)) are added.

Upon start of the process of displaying a fake level (step S411), the audio information acquiring section 28 acquires audio information from the broadcast signal 616 (step S422).

Next, the audio-to-text converting section 29 converts the audio information acquired by the audio information acquiring process (step S422) into character information (referred to as “audio-to-text conversion data”) (step S423).

In the present embodiment, a process of converting audio information into audio-to-text conversion data is carried out using AI audio analysis processing. Note that AI audio analysis processing by is a technology such as Siri in iPhones (registered mark), audio input technology provided by Google LLC, for example, of recognizing a hotword, “OK, Google”.

Next, the audio-to-text conversion data converted by the audio-to-text converting process (step S423) is stored in the nonvolatile memory 42 of the storage 4 by the audio-to-text conversion data storing section 30 (step S424).

Next, the audio-to-text conversion data stored by the audio-to-text conversion data storing process (step S424) is displayed on the display screen 711 (step S425).

In the case of television broadcasting, the audio-to-text conversion data is displayed on the display screen 711 by superimposing the audio-to-text conversion data on a screen of the television broadcasting, dividing the display screen 711 into two screens, or the like.

The audio-to-text conversion data converted into character information can be handled in the same manner as the case of display information according to the first embodiment.

The subsequent processes are the same as the processes of displaying a fake level according to the first embodiment, and accordingly, explanation therefor will be omitted.

According to the present embodiment, the advantageous effects of the present invention can also be obtained in receiving broadcasts such as television, radio, and the like.

In the present embodiment, user personality information (gullibility) is used as information about a user, whereas user physical condition information (state of excitement) described in the second embodiment may be used. Furthermore, both user personality information (gullibility) and user physical condition information (state of excitement) described in the third embodiment may be used.

As one of the applications of the present embodiment, the same process may be carried out on audio information (audio information file) provided in the WEB page 613, SNS 614, or the like acquired through the internet 612 as a communication network. Thus, the advantageous effects of the present invention can be obtained for the audio information acquired through the internet 612 as well.

Furthermore, extracting a character pattern from still image information or moving image information and converting it into character information naturally causes the advantageous effects of the present invention. For example, a television that is not connected to the internet 612 may be configured to receive a broadcast signal (broadcast radio wave), convert audio information into character information, and display a user-specific fake level or a warning as a subtitle on a screen of the television. In this case, such a television may be configured to receive user personality information by means of a setting operation, or estimate the age of a viewer (for example, whether the viewer is a child or an adult) or gender based on the programs that the viewer viewed in the past. For example, if a user's (viewer's) viewing time of programs for children is long, the possibility that the viewer is a child is high, and in this case, a user-specific fake level coefficient may be set to a value more than the normal value of 1.0.

In the embodiments described above, examples of using a smartphone have been described, whereas the present invention can be applied to information display devices other than the smartphone 1, for example, tablets, personal computer devices including notebook computers, AR (augmented reality) glasses, and HMDs (head-mounted displays).

Note that the fake level display function of the present invention should be selected to be turned on (enabled) or off (disabled) by a user since this function is to be additionally added to the function of displaying information to be displayed. In view of this, the fake level display function may be set to be on (enabled) or off (disabled) by any operation input device such as the main processor 2, the storage 4, the touch sensor 91, and the operation key 92 illustrated in FIG. 2. Furthermore, it may be configured that a user-specific fake level is displayed, together with display information, on the same display screen 711, or the user-specific fake level and the display information are displayed on the plurality of display screens 711, respectively, and the display screens 711 are switched. Furthermore, it may be configured that, while only the display information is displayed on the display screen 711 even if a user-specific fake level has been calculated, when a user starts SNS or the like on the smartphone 1 to transfer the display information, a warning for the transferring operation, which corresponds to the user-specific fake level, is displayed (or popped-up).

It should be noted that the present invention is not limited to the first to fourth embodiments which have been described as examples of embodiments of the present invention, and various modifications can be made. For example, a part of the configuration of one embodiment may be replaced with a part of other embodiments, and configurations of other embodiments may be added to configurations of a certain embodiment. All of these are included in the scope of the present invention. The numerical values and messages appearing in the text and drawings are merely examples, and thus the advantageous effects of the present invention are not impaired even if different ones are used.

Furthermore, the embodiments described above have been explained on the assumption that a single user uses a mobile information terminal as an information display device, whereas a plurality of users may use one mobile information terminal. In this case, user personality information, physical condition information, and the like are specific to each user, and thus they need to be processed separately and independently.

In the case where a plurality of users uses one mobile information terminal, storing personality information, physical condition information, and the like for each user in the nonvolatile memory 42 of the storage 4 in linkage with authentication of each user and using the stored information enables a user-specific fake level for each user to be displayed. As authentication of a user, a commonly used technology such as face authentication, fingerprint authentication, and password authentication may be used.

Thus, the advantageous effects of the present invention can be obtained even in the case of using one mobile information terminal by a plurality of users.

The functions and the like of the invention described above may be implemented in hardware by designing some or all of them, for example, in an integrated circuit. They may also be implemented in software by a microprocessor unit or the like interpreting and executing a program that realizes the respective functions, and the like. Hardware and software may be used together. The software may be stored in the ROM 41 and the like of the smartphone 1 in advance at the time of product shipment. It may be obtained from the external server 615, and the like on the internet 612 after product shipment. The software may also be obtained from the software provided on a memory card, optical disc, or the like.

Furthermore, the control lines and information lines which are considered to be necessary for the purpose of explanation are indicated herein, but not all the control lines and information lines of actual products are necessarily indicated. It may be considered that almost all the configurations are actually connected to each other.

### REFERENCE SIGNS LIST

1: smartphone
2: main processor
3: system bus
4: storage
10: user
11: controller
12: communication processor
13: display information acquiring section
14: display information storing section
15: fake-related information acquiring section
16: information fake level calculating section
17: information fake level data storing section
18: personality information acquiring section
19: personality information storing section
20: user-specific fake level coefficient calculating section
21: display data output section
22: physical condition measurement device detecting section
23: physical condition measurement value acquiring section
24: average value calculating section
25: physical condition information storing section
26: excitement state estimating section
27: excitement state information storing section
28: audio information acquiring section
29: audio-to-text converting section
30: audio-to-text conversion data storing section
41: ROM
42: nonvolatile memory
43: RAM
51: GPS receiver
52: geomagnetic sensors
53: acceleration sensors
54: gyro sensors
61: LAN communication I/F
62: telephone network communication I/F
63: near field wireless communication I/F
64: broadcast receiver
71: display
72: in-camera
73: out-camera
81: microphone
82: speaker
91: touch sensor
92: operation key
611: wireless LAN router
612: internet
613: WEB page
615: external server
616: broadcast signal
621: mobile phone base station
711: display screen
712: display information area
713: fake level area
714: user-specific fake level
721: physical condition measurement device

1. An information display device comprising:
a communication device connected to a communication network;
a display; and
a processor connected to each of the communication device and the display,
the processor being configured to:
calculate an information fake level indicating a level of credibility of display information that is received by the communication device via the communication network and displayed on the display based on a characteristic relating to the credibility on the display information;
calculate a user-specific fake level coefficient indicating a level in which a user who operates the information display device is able to judge accuracy of the credibility of the display information based on an index representing how the user tends to judge the credibility;
correct the information fake level using the user-specific fake level coefficient to calculate a user-specific fake level; and
display the user-specific fake level on the display.

2. The information display device according to claim 1, wherein the processor calculates the user-specific fake level coefficient based on personality information including gullibility of the user as the index.

3. The information display device according to claim 1, wherein the processor calculates the user-specific fake level coefficient based on physical condition information about the user as the index.

4. The information display device according to claim 3, further comprising a near field wireless communication device wirelessly connected to an external physical condition measurement device, wherein
the processor is connected to the near field communication device, and
the processor calculates the user-specific fake level coefficient based on the physical condition information about the user measured by the external physical condition measurement device.

5. The information display device according to claim 4, further comprising a storage in which the physical condition information about the user measured in the past by the external physical condition measurement device is stored, wherein
the processor is connected to the storage, and
the processor calculates the user-specific fake level coefficient based on a result obtained by comparing an average value of the physical condition information about the user measured in the past by the external physical condition measurement device with the physical condition information currently measured by the external physical condition measurement device.

6. The information display device according to claim 4, wherein the processor calculates the user-specific fake level coefficient based on both the physical condition information about the user measured by the external physical condition measurement device and the personality information including the gullibility of the user.

7. The information display device according to claim 3, wherein the processor estimates, as the physical condition information about the user, a state of excitement of the user using at least one of or any combination of a blood pressure, a body temperature, a respiration rate, a heart rate, and a sweat rate of the user, and calculates the user-specific fake level coefficient based on a result obtained by the estimation.

8. The information display device according to claim 1, wherein the processor displays the display information and the user-specific fake level on a same screen of the display.

9. The information display device according to claim 1, wherein, the processor uses, as the characteristic relating to the credibility on the display information, at least one or any combination of a type of a site where the display information was made available, a person who wrote the display information, information indicating a person who made the display information available, evaluation information in which the credibility of the display information has been evaluated and the evaluation is provided, when the display information was spread through public networks, how fast the display information was spread through public networks, and sentence expressions included in the display information.

10. An information display device comprising:
a communication device connected to a communication network;
a display; and
a processor connected to each of the communication device and the display,
the processor being configured to:
acquire an information fake level indicating a level of credibility of display information that is received by the communication device via the communication network and displayed on the display;
calculate a user-specific fake level coefficient indicating a level in which a user who operates the information display device is able to judge accuracy of the credibility of the display information based on an index representing how the user tends to judge the credibility;
correct the information fake level using the user-specific fake level coefficient to calculate a user-specific fake level; and
display the user-specific fake level on the display.

11. An information display device comprising:
a broadcast receiver that receives a broadcast signal including audio information;
a display; and
a processor connected to each of the broadcast receiver and the display,
the processor being configured to:
convert the audio information included in the broadcast signal received by the broadcast receiver into character information;
calculate an information fake level indicating a level of credibility of the character information based on a characteristic relating to the credibility on the audio information;
calculate a user-specific fake level coefficient indicating a level in which a user who operates the information display device is able to judge accuracy of the credibility of the character information;
correct the information fake level using the user-specific fake level coefficient to calculate a user-specific fake level; and
display the user-specific fake level on the display.

12. The information display device according to claim 11, wherein the processor uses, as the characteristic relating to the credibility on the audio information, at least one or any combination of a broadcasting station that broadcasted the audio information, evaluation information in which the credibility of the audio information has been evaluated and the evaluation is provided, when the audio information was spread through public networks, how fast the audio information was spread through public networks, and sentence expressions included in the audio information.

13. An information display method to be executed by an information display device including a communication device connected to a communication network, a display, and a processor connected to each of the communication device and the display, the processor executing the steps of:
calculating an information fake level indicating a level of credibility of display information that is received by the communication device via the communication network and displayed on the display based on a characteristic relating to the credibility on the display information;
calculating a user-specific fake level coefficient indicating a level in which a user who operates the information display device is able to judge accuracy of the credibility of the display information based on an index representing how the user tends to judge the credibility;
correcting the information fake level using the user-specific fake level coefficient to calculate a user-specific fake level; and
displaying the user-specific fake level on the display.

14. An information display method to be executed by an information display device including a broadcast receiver that receives a broadcast signal including audio information, a display, and a processor connected to each of the broadcast receiver and the display, the processor executing the steps of:
converting the audio information included in the broadcast signal received by the broadcast receiver into character information;
calculating an information fake level indicating a level of credibility of the character information based on a characteristic relating to the credibility on the audio information;
calculating a user-specific fake level coefficient indicating a level in which a user who operates the information display device is able to judge accuracy of the credibility of the character information based on an index representing how the user tends to judge the credibility;
correcting the information fake level using the user-specific fake level coefficient to calculate a user-specific fake level; and
displaying the user-specific fake level on the display.
